(19) United States
(12) Patent Application Publication
(10) Pub. No.: US 2005/0112093 A1
(43) Pub. Date: May 26, 2005

(54) Combination therapy for HCV infection

(76) Inventors: Ene Ette, Framingham, MA (US); John J. Alam, Cambridge, MA (US); Robert Stephen Kauffman, Chestnut Hill, MA (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/964,243

(22) Filed: Oct. 12, 2004

Related U.S. Application Data
(60) Provisional application No. 60/510,733, filed on Oct. 11, 2003.

(51) Int. Cl. A61K 38/21; A61K 38/05; A61K 31/7056

(52) U.S. Cl. 424/85.4; 514/43; 514/18

(57) ABSTRACT

The present invention relates to therapeutic combinations comprising VX-497, ribavirin, and interferon. The present invention also relates to methods using the therapeutic combinations of the present invention for treating HCV infection or alleviating one or more symptoms thereof in a patient. The present invention also provides kits comprising the combinations of the present invention.

### CROSS-REFERENCE TO RELATED APPLICATIONS

The present application claims the benefit under 35 U.S.C. § 119 of U.S. Provisional patent application No. 60/510,733, filed Oct. 11, 2003, the entire contents of the application being incorporated herein by reference.

### TECHNICAL FIELD OF THE INVENTION

The present invention relates to therapeutic combinations comprising VX-497, ribavirin, and interferon. The present invention also relates to methods using the therapeutic combinations of the present invention for treating HCV infection or alleviating one or more symptoms thereof in a patient. The present invention also provides kits comprising the therapeutic combinations of the present invention. The present invention also provides a pharmaceutical regimen for administering the therapeutic combinations of the present invention.

### BACKGROUND OF THE INVENTION

HCV is a RNA virus of the Flaviviridae family. Acute infection with HCV causes a generally mild, often asymptomatic, acute hepatitis. However, at least 85% of patients infected with HCV do not fully clear the virus and develop chronic infection of the liver. Once chronic hepatitis C is established, spontaneous clearance of the virus is rare and the majority of patients with chronic hepatitis C develop slowly progressive liver disease. Twenty years after infection, most patients have evidence of ongoing chronic hepatitis and at least 20% have cirrhosis. Long-term sequelae of chronic hepatitis C include cirrhosis, hepatic failure, and hepatocellular carcinoma. It is estimated that HCV infects 170 million persons worldwide. Over the next ten years, as a larger proportion of patients who are currently infected enter the third decade of their infection, the number of deaths attributed to hepatitis C is expected to significantly increase.

Typical symptoms of HCV infection include elevated ALT, positive test for anti-HCV antibodies, presence of HCV as demonstrated by a positive test for HCV-RNA, clinical stigmata of chronic liver disease, or hepatocellular damage.

Until 1999, the approved therapy in the European Union (EU) for chronic HCV infection was interferon alfa (IFN-α); e.g., Intron® A, Viraferon®, or Infergen®. The response rate was relatively poor with only 20% of patients achieving a sustained virological response (SVR) following six months of therapy. SVR is the number of patients with undetectable HCV RNA six months after discontinuation of treatment. The lack of durable antiviral response along with the need for injections, and the various side effects of the agent (including flu-like syndrome, nausea, anorexia, insomnia and depression) have limited the use of the therapy.

Ribavirin, a broad-spectrum antiviral agent, has reported activity in chronic hepatitis C. When used alone, ribavirin decreases liver enzyme levels in most patients during treatment. However, liver enzymes return to baseline values when treatment is discontinued. Additionally, ribavirin treatment only minimally and transiently decreases serum HCV RNA levels. More encouraging results have been obtained when ribavirin has been combined with IFN-α. In two large, controlled trials of the combination of ribavirin, 1000-1200 mg/day orally, and IFN-α, 3 MIU three times weekly subcutaneously, treatment naïve hepatitis C patients demonstrated statistically significant increased SVR for the combination, compared to IFN-α alone. Following 6 months of treatment, the SVR for combination therapy was 29-32%, compared to 6-17% (the latter representing 48 weeks of treatment) for IFN-α alone. A longer course of combination treatment, 48 weeks, resulted in a somewhat higher proportion of treatment naïve patients exhibiting a SVR (37-42%). The combination therapy received EMEA regulatory approval in 1999 and is marketed by Schering Plough Corporation. Limitations of ribavirin therapy include the development of drug-induced hemolytic anemia. A majority of patients demonstrate a mean decrease in hemoglobin of 2-3 g/dL over the course of treatment. Decreases in hemoglobin concentrations to less than 10 g/dL, necessitating a reduction in the dose of ribavirin, have been observed in approximately 8% of patients receiving combination therapy. ribavirin treatment has also been associated with nonspecific constitutional symptoms such as fatigue, insomnia, depression and vertigo. In the trials reported to date, a small proportion of patients receiving the combination of ribavirin and IFN-α have required dose reduction or treatment discontinuation for toxicity management, generally because of the hemolytic anemia. In addition, it has been suggested that hemolysis may damage the liver by increasing iron absorption.

In an attempt to further improve the SVR rates of the ribavirin and IFN-α combination, recent developments have focused on the interferon component of the therapy. Through a process termed pegylation, polyethylene glycol (PEG) molecules are covalently bound to the interferon protein. Pegylation results in an increased protein half-life consequential to reduced renal clearance and proteolysis. Pegylated interferon alfa (PEG-IFN-α) exhibits less variability in serum concentrations than standard IFN-α resulting in a more consistent antiviral pressure on the virus. Two different PEG-IFN-α products have been studied. PEG-IFN-α2a (Pegasys®; Roche Laboratories) incorporates 40 kDa PEG molecules with a resultant serum half-life of approximately 80 hours. PEG-IFN-α2b (PEG-Intron™ or ViraferonPEG™; Schering Plough Corporation) incorporates 12 kDa PEG molecules with a serum half-life of approximately 31 hours.

A phase III study has been completed in which the antiviral activity of PEG-IFN-α2a (Pegasys®)+ribavirin was evaluated following administration of a 48-week treatment course to treatment naïve hepatitis C patients. 1,149 patients were enrolled in the study and received either 180 μg PEG-IFN-α2a+ribavirin, 180 μg PEG-IFN-α2a+placebo or IFN-α2b+ribavirin. PEG-IFN-a2a+ribavirin demonstrated a statistically significant increase in SVR (56%) compared to PEG-IFN-α2a (30%) and IFN-α2b+ribavirin (45%). The combination of Pegasys® and ribavirin has not received FDA or EMEA regulatory approval at this time.

The antiviral activity of the PEG-IFN-α2b (PEG-Intron™/ViraferonPEG™)+ribavirin combination has been evaluated in a phase III study in treatment naïve patients. A total of 1,530 patients were enrolled in the study and randomized to one of three treatment arms; 1.5 μg/kg PEG-IFN-α2b subcutaneously once a week plus 800 mg/day ribavirin for 48 weeks, 1.5 μg/kg PEG-IFN-α2b subcutaneously once a week plus 1000-1200 mg/day ribavirin for four weeks followed by 0.5 μg/kg PEG-IFN-α2b subcutaneously once a week plus 1000-1200 mg/day ribavirin for 44 weeks, or IFN-α2b+ribavirin for 48 weeks. The SVR rates were 54%, 47%, and 47%, respectively. The combination of PEG-Intron™ and ribavirin received regulatory approval from the U.S. and E.U. regulatory authorities in 2001

Methods of treating HCV infection using ribavirin and an interferon alpha are disclosed in, e.g., U.S. Pat. No. 6,299,872, U.S. Pat. No. 6,387,365, U.S. Pat. No. 6,172,046, U.S. Pat. No. 6,472,373, the disclosures of which are incorporated herein by reference.

The combination of pegylated interferon and ribavirin, while effective, has drawbacks. The SVR rates in the region of 50-55%, coupled with toxicities of these two agents, point to the need for additional effective therapies with improved safety profiles. There is a need for an effective therapy for HCV infection that has reduced side effects and increased efficacy.

### SUMMARY OF THE PRESENT INVENTION

It is an object of the present invention to provide a therapeutic combination comprising VX-497, ribavirin, and interferon.

It is another object of the present invention to provide a method of treating HCV infection or alleviating one or more symptoms thereof in a patient, comprising administering to said patient a composition of the present invention.

It is yet another object of the present invention to provide a pharmaceutical regimen for treating HCV infection in a patient.

### DETAILED DESCRIPTION OF THE INVENTION

The following definitions are used herein:
“Peg-Intron” means PEG-Intron®, peginteferon alfa-2b, available from Schering Corporation, Kenilworth, N.J.;
“Intron” means Intron-A®, interferon alfa-2b available from Schering Corporation, Kenilworth, N.J.;
“ribavirin” means ribavirin (1-beta-D-ribofuranosyl-1H-1,2,4-triazole-3-carboxamide, available from ICN Pharmaceuticals, Inc., Costa Mesa, Calif.; described in the Merck Index, entry 8365, Twelfth Edition; also available as Rebetol® from Schering Corporation, Kenilworth, N.J., or as Copegus® from Hoffmann-La Roche, Nutley, N.J.;
“Pagasys” means Pegasys®, peginterferon alfa-2a available Hoffmann-La Roche, Nutley, N.J.;
“Roferon” mean Roferon®, recombinant interferon alfa-2a available from Hoffmann-La Roche, Nutley, N.J.;
“Berefor” means Berefor®, interferon alfa 2 available from Boehringer Ingelheim Pharmaceutical, Inc., Ridgefield, Conn.;
Sumiferon®, a purified blend of natural alpha interferons such as Sumiferon available from Sumitomo, Japan;
Wellferon®, interferon alpha n1 available from Glaxo_Wellcome LTd., Great Britain;
Alferon®, a mixture of natural alpha interferons made by Interferon Sciences, and available from Purdue Frederick Co., Conn.;
“VX-497” means a compound having the formula: 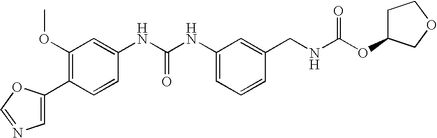
   or a pharmaceutically acceptable salt thereof.
VX-497 is a potent IMPDH inhibitor identified by Vertex Pharmaceuticals Inc., and described in U.S. Pat. No. 6,541,496.
“bid” means twice daily;
“tid” means thrice daily;
“qid” means four times each day;
“biw” means twice weekly;
“tiw” means thrice weekly.

The term “therapeutic combination” as used herein means a combination of one or more active drug substances, i.e., compounds having a therapeutic utility. Typically, each such compound in the therapeutic combinations of the present invention will be present in a pharmaceutical composition comprising that compound and a pharmaceutically acceptable carrier. The compounds in a therapeutic combination of the present invention may be administered simultaneously or separately, as part of a regimen.

According to one embodiment, the present invention provides a therapeutic combination comprising VX-497 and ribavirin.

According to another embodiment, the present invention provides a therapeutic combination comprising an IMPDH inhibitor such as VX-497, ribavirin, and interferon.

The first component of the therapeutic combination, namely, VX-497, is comprised in a composition. Such a composition comprises VX-497 (“the VX-497 composition”) and a pharmaceutically acceptable adjuvant or carrier.

Preferably, the VX-497 composition comprises VX-497 in an amount sufficient for a dosage of at least about 60 mg/day.

According to another embodiment, the VX-497 composition comprises VX-497 in an amount sufficient for a dosage of between about 60 mg/day to about 220 mg/day.

According to another embodiment, the VX-497 composition comprises VX-497 in an amount sufficient for a dosage of between about 60 mg/day to about 150 mg/day.

According to another embodiment, the VX-497 composition comprises VX-497 in an amount sufficient for a dosage of between 70 mg/day to about 120 mg/day.

According to another embodiment, the VX-497 composition comprises VX-497 in an amount sufficient for a dosage of between about 80 mg/day to about 100 mg/day.

According to another embodiment, the VX-497 composition comprises VX-497 in an amount sufficient for a dosage of about 85 mg/day to about 90 mg/day.

According to another embodiment, the VX-497 composition comprises VX-497 in an amount sufficient for a dosage of between about 90 mg/day to about 220 mg/day.

According to another embodiment, the VX-497 composition comprises VX-497 in an amount sufficient for a dosage of between about 90 mg/day to about 120 mg/day.

According to another embodiment, the VX-497 composition comprises VX-497 in an amount sufficient for a dosage of between 100 mg/day to about 110 mg/day.

According to another embodiment, the VX-497 composition comprises VX-497 in an amount sufficient for a dosage of about 100 mg/day.

According to another embodiment, the VX-497 composition comprises VX-497 in an amount sufficient for a dosage of between about 150 mg/day to about 220 mg/day.

According to another embodiment, the VX-497 composition comprises VX-497 in an amount sufficient for a dosage of between about 170 mg/day to about 210 mg/day.

According to another embodiment, the VX-497 composition comprises VX-497 in an amount sufficient for a dosage of between about 180 mg/day to about 210 mg/day.

According to another embodiment, the VX-497 composition comprises VX-497 in an amount sufficient for a dosage of between about 200 mg/day.

According to one embodiment, the VX-497 composition comprises VX-497 in a formulation suitable for dosing once a day, bid, tid, qid, five times a day, six times a day. For example, if a VX-497 composition comprises about 100 mg/day dosage of VX-497, and a bid dosing is desired, then the VX-497 composition will comprise VX-497 in a formulation, e.g., a tablet, containing about 50 mg of VX-497

According to another embodiment, the VX-497 composition comprises VX-497 in a formulation suitable for dosing bid.

Or, the VX-497 composition comprises VX-497 in a formulation suitable for dosing tid.

According to another embodiment, the VX-497 composition comprises VX-497 in an amount sufficient for a dosage of between about 90 mg/day to about 120 mg/day wherein said VX-497 is formulated for dosing bid.

According to another embodiment, the VX-497 composition comprises VX-497 in an amount sufficient for a dosage of between 100 mg/day to about 110 mg/day, wherein said VX-497 is formulated for dosing bid.

According to another embodiment, the VX-497 composition comprises VX-497 in an amount sufficient for a dosage of about 100 mg/day, wherein said VX-497 is formulated for dosing bid.

In the therapeutic combinations of the present invention, VX-497 may be replaced by other IMPDH inhibitors known in the art. Preferably, such other IMPDH inhibitors are used in dosage amounts that provide an equivalent exposure level in a patient (e.g., in serum plasma) when compared to the exposure level of the corresponding VX-497 dosage amount. Examples of such other IMPDH inhibitors include, e.g., Cellcept, VX-944, VX-148, and mizorubin.

The second component of the therapeutic combination, namely ribavirin, is comprised in a composition (“ribavirin composition”). Typically, such a composition comprises ribavirin and a pharmaceutically acceptable adjuvant or carrier.

According to one embodiment, the ribavirin composition comprises ribavirin in an amount sufficient for a dosage of between 400 mg/day to about 1200 mg/day.

According to another embodiment, the ribavirin composition comprises ribavirin in an amount sufficient for a dosage of between about 800 mg/day to about 1200 mg/day.

According to another embodiment, the ribavirin composition comprises ribavirin in an amount sufficient for a dosage of between about 1000 mg/day to about 1200 mg/day.

According to another embodiment, the ribavirin composition comprises ribavirin in an amount sufficient for a dosage of about 1000 mg/day or about 1200 mg/day.

According to another embodiment, the ribavirin composition comprises ribavirin in an amount sufficient for dosage of between about 300 mg/day to about 800 mg/day, more preferably, between about 300 mg/day to about 700 mg/day. Or, more preferably, it is present in an amount sufficient for a dosage of between 500 mg/day to about 700 mg/day. Or, more preferably, it is present in an amount sufficient for a dosage of between 400 mg/day to about 600 mg/day.

According to a yet another embodiment, ribavirin is either Rebetol® or Copegus®.

According to one embodiment, the ribavirin composition comprises ribavirin in a formulation suitable for dosing once a day, bid, tid, qid, five times a day, or six times a day. For example, if a therapeutic combination comprises about 1000 mg/day dosage of ribavirin, and a dosing of five times a day is desired, then the therapeutic combination will comprise ribavirin in a formulation, e.g., a tablet, containing, e.g., about 200 mg of ribavirin.

Or, the ribavirin composition comprises ribavirin in a formulation suitable for dosing at least bid. More preferably, ribavirin is formulated for dosing bid, tid, qid, or five times a day. Preferably, ribavirin is formulated for dosing bid or tid a day. More preferably, ribavirin is formulated for dosing bid.

The term “interferon” as used herein means a member of a family of highly homologous species-specific proteins that inhibit viral replication and cellular proliferation, and modulate immune response, such as interferon alpha, interferon beta, or interferon gamma. The Merck Index, entry 5015, Twelfth Edition.

According to another embodiment, the therapeutic combination of the present invention utilizes natural alpha interferon 2a. Or, the therapeutic combination of the present invention utilizes natural alpha interferon 2b. Preferably, the therapeutic combination of the present invention utilizes recombinant alpha interferon 2a or 2b. More preferably, the interferon is pegylated alpha interferon 2a or 2b. Interferons suitable for the present invention include:
(a) Intron,
(b) Peg-Intron,
(c) Pegasys,
(d) Roferon,
(e) Berofor,
(f) Sumiferon,
(g) Wellferon,
(h) consensus alpha interferon available from Amgen, Inc., Newbury Park, Calif.,
(i) Alferon;
(j) Viraferon®;
(k) Infergen®.

According to another embodiment, the therapeutic combination comprises VX-497 and an interferon selected from Intron, Peg-Intron, Pegasys, Roferon, Berofor, Sumiferon, Wellferon, consensus alpha interferon, or Alferon.

According to another embodiment, the therapeutic combination comprises VX-497 in one of Intron, Roferon, Peg-Intron, or Pegasys.

According to another embodiment, the therapeutic combination comprises Intron or Roferon in an amount sufficient for a dosage of about 4 million IU to about 12 million IU per week. Preferably, Intron or Roferon is present in an amount sufficient for a dosage of about 6 million IU to about 10 million IU. Yet more preferably, Intron or Roferon is present in an amount sufficient for a dosage of about 8 million IU to about 9 million IU. More preferably, Intron or Roferon is present in an amount sufficient for a dosage of about 9 million IU.

The amount of Peg-Intron or Pegasys in the therapeutic combination of the present invention depends on the body weight of the patient being treated.

According to one embodiment, the therapeutic combination comprises Peg-Intron or Pegasys in an amount sufficient for a dosage of between about 0.5 μg/kg/week to about 2 μg/kg/week. According to another embodiment, Peg-Intron or Pegasys is present in an amount sufficient for a dosage of between about 1 μg/kg/week to about 2 μg/kg/week. Or, Peg-Intron or Pegasys is present in an amount sufficient for a dosage of about 1.5 μg/kg/week.

Pharmaceutical carriers and adjuvants useful for formulating each of VX-497 and ribavirin are known in the art. Formulations comprising VX-497 are disclosed in U.S. Pat. No. 6,541,496, the disclosure of which is incorporated herein by reference. Formulations comprising ribavirin are disclosed in U.S. Pat. No. 4,211,771.

According to another embodiment, the present invention provides kits for use in treating HCV infection in a patient. The kits of the present invention comprise any one of the therapeutic combinations of the present invention. The kits further comprise instructions for utilizing the therapeutic combinations. The kits may be tailored to the needs of classes or types of patients or other clinically relevant factors such as age, body weight, concomitant diseases/conditions, severity and stage of HCV infection, responsiveness or non-responsiveness to prior treatments, propensity for side effects, etc. For example, the therapeutic combination in a kit may be tailored for dosages suitable for patients having a body weight of, e.g., 75 kg. Or, the therapeutic combination in a kit may be tailored for dosages suitable for patients have a body weight of, e.g., less than or equal to 75 kg. Or, the therapeutic combination in a kit may be tailored for pediatric use, wherein the dosage for children is varied depending on factors such as age, body weight, severity of disease, etc.

According to another embodiment, the present invention provides a kit comprising:
(i) a plurality of VX-497 compositions;
(ii) a plurality of ribavirin compositions;
(iii) a plurality of interferon compositions; and
(iv) instructions for utilizing above compositions.

According to another embodiment, the kit comprises VX-497 compositions, wherein each composition contains a dosage amount of VX-497 according to any one of the embodiments hereinabove. In one embodiment, each said composition contains at least, and preferably, about 50 mg of VX-497. In one embodiment, each said composition contains at least, and preferably, about 1000 mg of VX-497

According to another embodiment, the kit comprises ribavirin compositions, wherein each composition contains a preferred dosage amount of ribavirin as described above. According to another embodiment, each said composition contains about 200 mg of ribavirin. Preferably, each said composition contains about 200 mg of ribavirin in a capsule formulation.

According to another embodiment, the kit comprises interferon alpha compositions wherein each composition contains a dosage amount of interferon as described above. Preferably, the interferon in the kit is Intron, Peg-Intron, Roferon, or Pegasys. More preferably, the interferon is Peg-Intron or Pegasys.

According to another embodiment, the kit comprises interferon alpha formulation in a single dose vial or a multiple dose vial. Preferably, the interferon alpha is in a formulation suitable for injection.

According to another embodiment, the present invention provides a method of treating HCV infection or alleviating one or more symptoms thereof in a patient comprising the step of administering to the patient a therapeutic combination according to the present invention. According to one embodiment, the patient has HCV genotype 1 infection.

According to another embodiment, the method of the present invention is useful in treating HCV infection or alleviating one or more symptoms thereof in a treatment naive patient, i.e., a patient who has not received any prior treatment for HCV infection.

According to another embodiment, the method of the present invention is useful in treating HCV infection or alleviating one or more symptoms thereof in a patient who is non-responsive to interferon monotherapy.

According to another embodiment, the method of the present invention is useful in treating HCV infection or alleviating one or more symptoms thereof in a patient who is non-responsive to a combination therapy using ribavirin and an interferon.

According to an alternative embodiment the present invention provides a method of reducing HCV-RNA levels in a patient in need thereof, comprising the step of administering to said patient a therapeutic combination according to the present invention. Preferably, the method of the present invention reduces the HCV-RNA levels in a patient to a less than detectable level.

A detectable level of HCV RNA as used in the present invention means at least 100 HCV RNA copies per ml of serum of a patient as measured by quantitative, multi-cycle reverse transcriptase PCT methodology. Such methods are well known in the art.

According to another embodiment, the present invention provides a pharmaceutical regimen, comprising administering to a patient in need thereof a therapeutic combination according to the present invention for at least 12 weeks. In one embodiment, the pharmaceutical regimen comprises administering to a patient in need thereof a therapeutic combination for between about 12 weeks and about 24 weeks. Or, the therapeutic combination is administered for at least 24 weeks. According to an alternate embodiment, the therapeutic combination is administered until the HCV RNA level in the patient is below a detectable level.

According to a another embodiment, the pharmaceutical regimen comprises administering to a patient in need thereof for at least about 12 weeks:
(i) a therapeutically effective amount of VX-497 bid;
(ii) a therapeutically effective amount of ribavirin bid;
(iii) a therapeutically effective amount of interferon alpha once a week.

According to one embodiment, VX-497 is dosed at least 40 mg bid. Or, VX-497 is dosed at between about 40 mg bid to about 120 mg bid. In another embodiment, VX-497 is dosed at about 50 mg bid. In yet another embodiment, VX-497 is dosed at about 100 mg bid.

According to another embodiment, ribavirin dosage is selected from 400 mg/day, 600 mg/day, 800 mg/day, 1000 mg/day, or 1200 mg/day, wherein each daily dosage is divided into a plurality of administrations during each day. Preferred dosages for each of such plurality of administrations during each day are 200 mg, 300 mg, 400 mg, 500 mg, or 600 mg.

According to another embodiment, interferon alpha is administered once a week. Preferably, Intron or Roferon is administered once a week. Or, a pegylated interferon is administered once a week. Preferred pegylated interferons include Peg-Intron or Pegasys. Preferred dosage amounts for interferon alpha in the pharmaceutical regimen are as described above.

### EXAMPLE

A 24-week, double blind, randomized, placebo controlled study was conducted on 31 patients that were non-responders to ribavirin/Peg-Intron therapy. The patients were divided into three groups. All three groups received ribavirin/Peg-Intron therapy. One group was administered a placebo, while a second group was administered VX-497 25 mg bid. The third group was administered VX-497 in a dosage according to the present invention.

More than 80% of patients in the third group, who received ribavirin, Peg-Intron, and VX-497 in dosages according to the present invention, achieved undetectable levels of HCV RNA at the end of 24 weeks.

1. A therapeutic combination comprising VX-497 and ribavirin.

2. A therapeutic combination comprising VX-497, ribavirin, and interferon.

3. The therapeutic combination according to claim 2, wherein said interferon is interferon alpha 2a.

4. The therapeutic combination according to claim 3, comprising VX-497 in an amount sufficient for a dosage of at least about 60 mg/day.

5. The therapeutic combination according to claim 4, comprising VX-497 in an amount sufficient for a dosage of between about 60 mg/day to about 220 mg/day.

6. The therapeutic combination according to claim 5, comprising VX-497 in an amount sufficient for a dosage of between about 60 mg/day to about 150 mg/day.

7. The therapeutic combination according to claim 6, comprising VX-497 in an amount sufficient for a dosage of between 70 mg/day to about 120 mg/day.

8. The therapeutic combination according to claim 7, comprising VX-497 in an amount sufficient for a dosage of between about 80 mg/day to about 100 mg/day.

9. The therapeutic combination according to claim 8, comprising VX-497 in an amount sufficient for a dosage of about 85 mg/day to about 90 mg/day.

10. The therapeutic combination according to claim 4, comprising VX-497 in an amount sufficient for a dosage of between about 90 mg/day to about 220 mg/day.

11. The therapeutic combination according to claim 10, comprising VX-497 in an amount sufficient for a dosage of between about 90 mg/day to about 120 mg/day.

12. The therapeutic combination according to claim 11, comprising VX-497 in an amount sufficient for a dosage of between about 100 mg/day to about 110 mg/day.

13. The therapeutic combination according to claim 12, comprising VX-497 in an amount sufficient for a dosage of about 100 mg/day.

14. The therapeutic combination according to claim 4, comprising VX-497 in an amount sufficient for a dosage of between about 150 mg/day to about 220 mg/day.

15. The therapeutic combination according to claim 14, comprising VX-497 in an amount sufficient for a dosage of between about 180 mg/day to about 210 mg/day.

16. The therapeutic combination according to claim 15, comprises VX-497 in an amount sufficient for a dosage of about 200 mg/day.

17. The therapeutic combination according to claim 4, comprising VX-497 in a formulation suitable for dosing once a day, bid, tid, qid, five times a day, six times a day.

18. The therapeutic combination according to claim 17, comprising VX-497 in a formulation suitable for dosing bid.

19. The therapeutic combination according to claim 17, comprising VX-497 in a formulation suitable for dosing tid.

20. The therapeutic combination according to claim 17, comprising VX-497 in an amount sufficient for a dosage of between about 90 mg/day to about 120 mg/day wherein said VX-497 is formulated for dosing bid.

21. The therapeutic combination according to claim 20, comprising VX-497 in an amount sufficient for a dosage of between 100 mg/day to about 110 mg/day, wherein said VX-497 is formulating for dosing bid.

22. The therapeutic combination according to claim 21, comprising VX-497 in an amount sufficient for a dosage of about 100 mg/day, wherein said VX-497 is formulated for dosing bid.

23. The therapeutic combination according to claim 4, comprising ribavirin in an amount sufficient for a dosage of between 400 mg/day to about 1200 mg/day.

24. The therapeutic combination according to claim 23, comprising ribavirin in an amount sufficient for a dosage of between about 800 mg/day to about 1200 mg/day.

25. The therapeutic combination according to claim 24, comprising ribavirin in an amount sufficient for a dosage of between about 1000 mg/day to about 1200 mg/day.

26. The therapeutic combination according to claim 25, comprising ribavirin in an amount sufficient for a dosage of about 1000 mg/day or about 1200 mg/day.

27. The therapeutic combination according to claim 26, comprising ribavirin in an amount sufficient for dosage of between about 300 mg/day to about 800 mg/day, more preferably, between about 300 mg/day to about 700 mg/day.

28. The therapeutic combination according to claim 27, comprising ribavirin in an amount sufficient for a dosage of between 500 mg/day to about 700 mg/day.

29. The therapeutic combination according to claim 28, comprising ribavirin in an amount sufficient for a dosage of between 400 mg/day to about 600 mg/day.

30. The therapeutic combination according to claim 4, wherein said ribavirin is Rebetol® or Copegus®.

31. The therapeutic combination according to claim 4, comprising ribavirin in a formulation suitable for dosing once a day, bid, tid, qid, five times a day, or six times a day.

32. The therapeutic combination according to claim 31, comprising ribavirin in a formulation suitable for dosing at least bid.

33. The therapeutic combination according to claim 4, comprising an interferon alpha 2a.

34. The therapeutic combination according to claim 33, wherein said interferon is selected from:
(a) Intron,
(b) Peg-Intron,
(c) Pegasys,
(d) Roferon,
(e) Berofor,
(f) Sumiferon,
(g) Wellferon,
(h) consensus alpha interferon;
(i) Alferon;
(j) Viraferon®; or
(k) Infergen®.

35. The therapeutic combination according to claim 34, wherein said interferon is selected from Intron, Peg-Intron, Pegasys, Roferon, Berofor, Sumiferon, Wellferon, consensus alpha interferon, or Alferon.

36. The therapeutic combination according to claim 34, wherein said interferon is Intron, Roferon, Peg-Intron, or Pegasys.

37. The therapeutic combination comprises according to claim 36, wherein Intron or Roferon is present in an amount sufficient for a dosage of about 4 million IU to about 12 million IU per week.

38. The therapeutic combination comprises according to claim 37, wherein Intron or Roferon is present in an amount sufficient for a dosage of about 6 million IU to about 10 million IU.

39. The therapeutic combination comprises according to claim 38, wherein, Intron or Roferon is present in an amount sufficient for a dosage of about 8 million IU to about 9 million IU.

40. The therapeutic combination comprises according to claim 39, Intron or Roferon is present in an amount sufficient for a dosage of about 9 million IU.

41. The therapeutic combination according to claim 36, wherein Peg-Intron or Pegasys is present in an amount sufficient for a dosage of between about 0.5 μg/kg/week to about 2 μg/kg/week.

42. The therapeutic combination according to claim 41, wherein Peg-Intron or Pegasys is present in an amount sufficient for a dosage of between about 1 μg/kg/week to about 2 μg/kg/week.

43. The therapeutic combination according to claim 42, wherein Peg-Intron or Pegasys is present in an amount sufficient for a dosage of about 1.5 μg/kg/week.

44. A kit comprising:
(i) a therapeutic combination according to claim 4; and
(ii) instructions for utilizing said combination.

45. The kit according to claim 44, comprising:
(i) a plurality of VX-497 formulations;
(ii) a plurality of ribavirin formulations;
(iii) a plurality of interferon formulations; and
(iv) instructions for utilizing said formulations.

46. A method of treating HCV infection or alleviating one or more symptoms thereof in a patient comprising the step of administering to the patient a therapeutic combination according to according to claim 4.

47. The method according to claim 46, wherein the HCV infection is genotype.

48. The method according to claim 46 or 47, wherein said patient is a treatment naive patient.

49. The method according to claim 46 or 47, wherein said patient is non-responsive to interferon monotherapy.

50. The method according to claim 46 or 47, wherein said patient who is non-responsive to a combination therapy using ribavirin and an interferon.

51. A method of reducing HCV-RNA levels in a patient in need thereof, comprising the step of administering to said patient a therapeutic combination according to anyone of claims 1-4.

52. The method according to claim 51, wherein said HCV-RNA levels in a patient are reduced to a less than detectable level.

53. A pharmaceutical regimen, comprising administering to a patient in need thereof a therapeutic combination according to claim 4 until the HCV RNA level in the patient is below a detectable level.

54. The pharmaceutical regimen according to claim 53, wherein said therapeutic combination is administered for at least 12 weeks.

55. The pharmaceutical regimen according to claim 53, wherein said therapeutic combination is administered for at least 24 weeks.

56. The pharmaceutical regimen according to claim 53, comprising administering to a patient in need thereof for at least about 12 weeks.

57. The pharmaceutical regimen according to claim 53, comprising administering to a patient in need thereof VX-497 bid; ribavirin bid; and interferon alpha once a week.

58. The pharmaceutical regimen according to claim 57, wherein VX-497 is dosed at least 40 mg bid.

59. The pharmaceutical regimen according to claim 58, wherein VX-497 is dosed at between about 40 mg bid to about 120 mg bid.

60. The pharmaceutical regimen according to claim 59, wherein said ribavirin dosage is selected from 400 mg/day, 600 mg/day, 800 mg/day, 1000 mg/day, or 1200 mg/day, wherein each daily dosage is divided into a plurality of administrations during each day.

61. The pharmaceutical regimen according to claim 57, wherein said interferon alpha is administered once a week.

62. The pharmaceutical regimen according to claim 61, wherein said interferon alpha is Intron or Roferon.

63. The pharmaceutical regimen according to claim 61, wherein said interferon alpha is a pegylated interferon.

64. The pharmaceutical regimen according to claim 63, wherein said pegylated interferon is Peg-Intron or Pegasys.
